(19) United States
(12) Patent Application Publication
(10) Pub. No.: US 2025/0073063 A1
(43) Pub. Date: Mar. 6, 2025

(54) Cartridge based spray applicator for ophthalmic medicine

(71) Applicant: Nanodropper, Inc., Rochester, MN (US)

(72) Inventor: Elias Lee Baker, Rochester, MN (US)

(21) Appl. No.: 18/819,916

(22) Filed: Aug. 29, 2024

Related U.S. Application Data
(60) Provisional application No. 63/535,938, filed on Aug. 31, 2023.

(51) Int. Cl. A61F 9/00

(52) U.S. Cl. CPC A61F 9/0026

(57) ABSTRACT

A cartridge-based device for providing a spritz of medication to a user's eyeball, the device supporting a sealed cartridge comprising at least two metered doses of medication wherein a moveable plug inside the device is actuated to force the medication through an ejection nozzle of the cartridge in the form of a spritz.

### BACKGROUND

The present invention relates to eye medication delivery systems, and more specifically to a device configured to provide a spray of liquid medication directly to a user's cornea.

### SUMMARY

An aspect of the present disclosure relates to a liquid ejection system for delivery of a liquid suspension or solution of medication to a user's eye. The system comprises a housing having an interior cavity; one or more cartridges having a reservoir for holding the liquid suspension or solution of medication, wherein the one or more cartridges are selectively loadable into to the interior cavity of the housing; a movable plug positioned within the interior cavity of the housing and configured to move into the one or more cartridges to eject the liquid suspension or aqueous solution of medication from the reservoir; wherein the one or more cartridges each have an ejection nozzle and wherein the liquid suspension or aqueous solution of medication is ejected from the cartridge and the housing through the ejection nozzle, and wherein the housing supports an alignment mechanism for automatically aligning a user's eye with the ejection nozzle.

The system further includes an actuator for initiating movement of the movable plug for ejecting the liquid suspension or aqueous solution of medication from the cartridge.

In one or more embodiments, the actuator is a linear actuator.

In one or more embodiments, the actuator is a piezoelectric actuator.

The alignment mechanism comprises a framed structure that is rigid or flexible and is attached to the housing that contacts the surface of the face of the user wherein the structure doesn't block the solution that is ejected out of the nozzle

The housing further supports a speaker thereon and electrical components for the speaker within the interior cavity and wherein the system is configured to provide an audible indicator or instruction for actuation of the system.

The housing further supports a light source for providing a visual indicator for alignment or focus for actuation of the system.

Each of the one or more cartridges are disposable.

Each of the one or more cartridges comprise at least one, two or more metered doses of medication for dosing each eye before replacement of the cartridge.

Another aspect of the present disclosure relates to a cartridge-based device for providing a spritz of medication to a user's eyeball, the device supporting a sealed cartridge comprising at least two metered doses of medication wherein a moveable plug inside the device is actuated to force the medication through an ejection nozzle of the cartridge in the form of a spritz.

The moveable plug provides the seal for the cartridge and wherein the movable plug facilitates the volumetric expansion and contraction of the medication allowing the cartridge to withstand various temperature and pressure changes as dictated by storage conditions, while maintaining the sterility of the medication therein.

The device includes an alignment mechanism configured to contact the surface of the user's face wherein the alignment mechanism spaces the ejection nozzle from the surface of the eye while maintaining alignment of the nozzle with the eye.

The alignment mechanism comprises one or more sensors for detecting contact with the surface of the user's face for initiating actuation and movement of the movable plug.

### BRIEF DESCRIPTION OF THE DRAWINGS

FIG. 1 is an isometric view of a cartridge based liquid ejection system according to one or more embodiments described herein.

FIG. 2 is a front view of the cartridge based liquid ejection system.

FIGS. 3A and 3B illustrate a section view of the cartridge based liquid ejection system about section A-A shown in FIG. 3A.

FIG. 4 is a top view of the cartridge based liquid ejection system.

FIG. 5 is a front view of a cartridge for the cartridge based liquid ejection system.

FIG. 6 is an isometric view of the cartridge.

FIG. 7 a cross-sectional view of a plunger in the cartridge for ejecting the liquid from the system.

FIG. 8 is a bottom view of the cartridge.

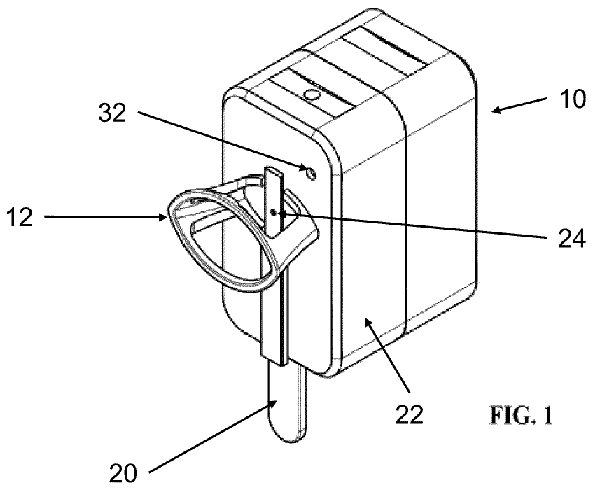

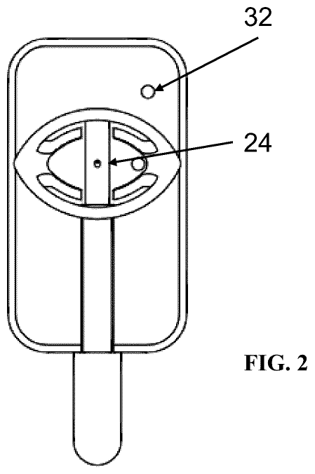

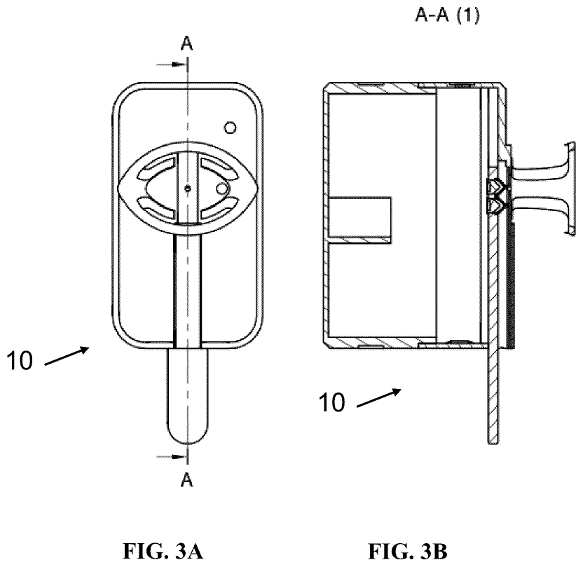

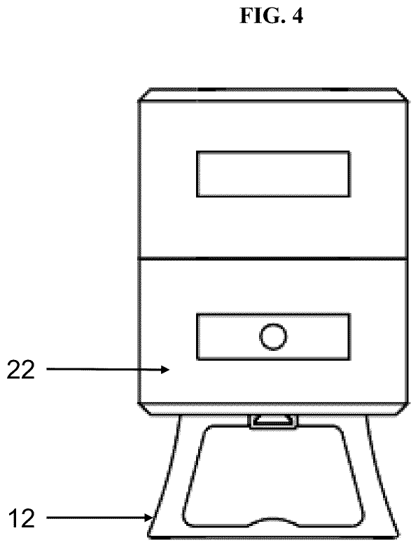

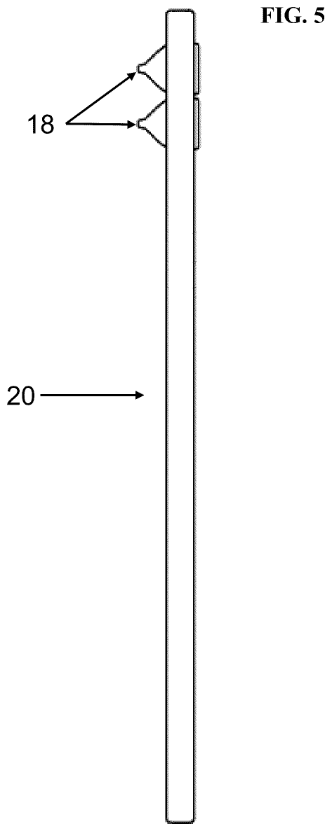

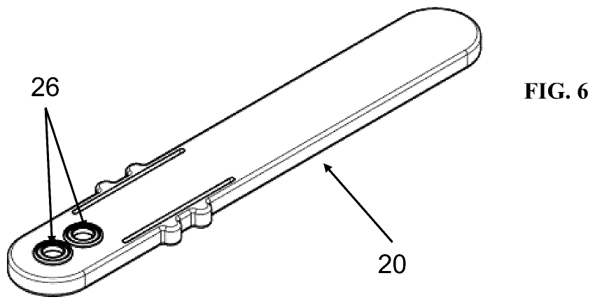

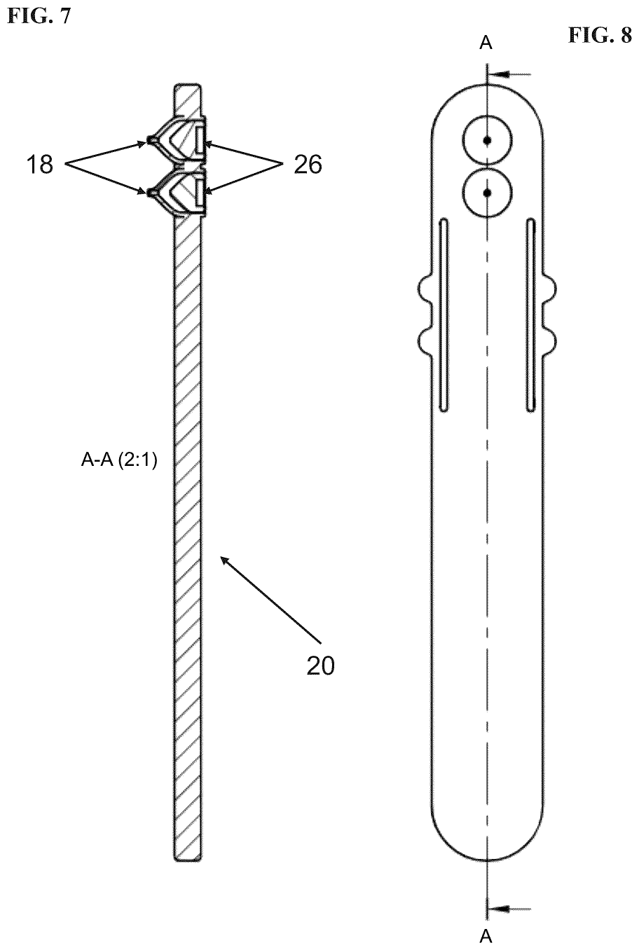

### DETAILED DESCRIPTION

A cartridge-based liquid ejection system for delivery of a liquid suspension or aqueous solution of medication to a user's eye(s) is described herein. In one or more embodiments, the suspension or solution may be delivered in the form of a spray, spray of microdroplets, aerosol, or the like. The device may further be configured to aid a user in aligning and timing the device for delivery of a spray or stream of medication directly to a user's eye.

The device includes a housing which is configured for a user to hold near and/or in front of an eye for delivery of a metered dose of medicine directly to the eye. The housing may have one or more alignment mechanisms which allow a user to easily position the housing a safe and effective distance from the eye along with alignment sufficient to deliver the spray of medicine to the eye ball. Such alignment mechanisms may be adjustable and selectively positional allow the same device to be used with both eyes and by users have various facial feature spacing. Additional mechanisms may include audible cues and/or visual cues for a user to focus and/or open their eye at the appropriate time for delivery of the medicine.

In one or more embodiments, the device is configured to accept one or more cartridges wherein the cartridges contain the liquid medicine. The type of medicine may be any liquid, solution or suspension or other material, for example, those medicines typically indicated for application to the user's eye.

The cartridges may be single use cartridges having a reservoir for holding the medicine prior to use. For example, the cartridges may be sealed and provided pre-filled with a single or multiple dose of medicine therein. These doses may be metered doses. Additionally, or alternatively, the cartridge may comprise multiple reservoirs such that each reservoir holds a dose of medicine, such as for each eye and/or for all or a portion of the dosages required to treat a condition. Similarly, the device may be configured to hold a plurality of cartridges, for example, a cartridge for each eye. It is also contemplated that the cartridges may be filled by a user from a larger supply of medicine and thus the device can be used to deliver eye drops of any formulation to a user's eye in the form of a fine spray, stream, or spritz or the like rather than a single drop.

The system further comprises a loading mechanism for installing or otherwise loading the one or more cartridges into the device. As the cartridges may be small given the micro-volume dose(es) of medicine contained therein, the cartridges may be more easily loaded with use of a loading mechanism. In one or more embodiments, a handle may be provided with a first end configured to support a cartridge thereon and the handle is then insertable into the device to deliver the cartridge to an installation position for use. The handle may be integrated with the cartridge or removable from the device leaving the cartridge behind as the cartridge engages with a catch mechanism inside the device to hold the cartridge in place for use. The handle may be disposable or reusable.

In one or more embodiments the system for delivery of a liquid suspension or aqueous solution of medication to a user's eye provides the medication in the form of a spray, fine spray, micro-droplet spray, spritz, aerosol or the like. The medicine may be ejected from the device with or without the use of a propellant such as a gas (e.g., pressurized air), and thus using one or more various mechanisms for ejecting the medicine as described in further detail below.

The housing of the system described herein comprises and/or supports a mechanism for ejecting the medicine. In one or more embodiments the mechanism for ejecting the medicine from the cartridge a piezo-based mechanism and may comprise a piezoelectric actuator for effecting ejection of the fluid from a cartridge reservoir in the form of a fine spray.

In one or more embodiments the mechanism for ejecting the medicine comprises a plunger element. The plunger is actuated to extend into the cartridge and force the medicine out of the cartridge through an opening in the cartridge, referred to as a nozzle, that is configured in combination with the plunger force and fluid pressure in the cartridge to produce a controlled and metered fine spray of medicine.

In one or more embodiments, as illustrated in the figures, a device 10 has three mechanisms that work together to deliver the medication. The first is a positioning frame 12 that rests on the user's face. The positioning frame 12 is primarily made of a stiff but flexible material. The positioning frame 12 aligns the nozzle 18 of a cartridge 20 inserted into the device 10 with the user's eye.

In one or more embodiments, the positioning frame may have a pressure or capacitive sensor embedded in the frame, for example near the end of the frame, that detects when the frame is in contact with the user's face. This sensor can activate the device for ejecting medicine and prime it for use.

A second element of one or more embodiments is the cartridge 20. The device 12 may be a cartridge-based liquid ejection system provided with disposable cartridges 20 that store the medication and provide the proper geometry to eject the suspension or solution onto the user's eyes. The cartridge 20 has a main body that is made of plastic materials that are known to be compatible with various medications. The cartridge 20 illustrated is provided with a minimum of two doses of medication, one for each eye. The cartridge 20 is configured to store the medication in an enclosed body and comes with a removable seal at the exit nozzle 18 to prevent contamination when not in use. The user removes the seal when ready to use the device 12 and before loading the cartridge 20 into the device's main body 22. The cartridge 20 has a flexible and movable plug 26 that forces liquid out the nozzle 18 when pushed by an external force. The movable plug 26 is designed to facilitate the medication's volumetric expansion and contraction, accommodating various temperature and pressure changes as dictated by storage conditions, while maintaining the sterility of its contents. The cartridge 20 is inserted to position inside the main body 22 and ensures that the nozzle 18 is aligned with an exit orifice 24 of the main body 22 of the housing.

A third mechanism of the device 10 is an actuator 30 that applies force to the plug 26 on the cartridge 20. This actuator 30 can be a pneumatic, electric, or mechanical type actuator. The velocity and displacement of the actuator, for example an actuator arm is programmed to allow sufficient force to be applied to the cartridge plug to enable the proper exit of the liquid medication from the cartridge and onto the user's eye. Additionally, or alternatively the nozzle of the cartridge is further configured to form a spray or stream of droplets or otherwise cooperate with the cartridge and movable plug and/or actuator to generate a spray.

In one or more embodiments, the actuator is a linear actuator which forces linear movement of the plug into the cartridge. Actuators include but are not limited to linear electrical actuators, piezo linear actuators, moving magnet actuators and combinations thereof. Additionally, or alternatively, actuators may or may not require the movable plug for ejecting the fluid as a spray or a spritz.

Additional or alternative actuators may comprise an impeller system for generating an air flow with the fluid or vibrating mesh nebulizers which operate on a “once-through” pumping system through micro-orifices powered by piezoelectrics and so solute concentration effects and damage to molecules are minimized.

In one or more embodiments, the cartridge based liquid ejection system may have a camera and LED light 32 attached to the body of the device. The camera may be used to detect proper dosing of medication and provide data to a software suite that can help the user track their medication usage. The camera can detect the placement of the device and help the user align the device properly to ensure successful administration of medication on the surface of the eye. The LED light will be used as a status indicator that shows the user various device information. This includes when the device is ready to be used, and when the device needs to be recharged. The LED light may be positioned away from the nozzle to be used as a distraction light to prevent a blink reflex in response to seeing the stream or spray of medication. This is to ensure successful administration of medication on the surface of the eye.

In one or more embodiments, the main body includes a push or haptic button that activates the actuator and thus moves the movable plug and ejects the liquid medication onto the user's eye.

In one or more embodiments, the main body may have a speaker that will respond to the various sensors and/or guide the user through the medication administration process.

In one or more embodiments, the main body can also have a protective cover that prevents outside foreign debris from contaminating the cartridge nozzles. The cover can be removed prior to using the device.

Although the present disclosure has been described with reference to preferred embodiments, workers skilled in the art will recognize that changes may be made in form and detail without departing from the spirit and scope of the disclosure.

1. A liquid ejection system for delivery of a liquid suspension, emulsion, or aqueous solution of medication to a user's eye comprising of:
a housing having an interior cavity;
one or more cartridges having a reservoir for holding the liquid suspension or aqueous solution of medication, wherein the one or more cartridges are selectively loadable into to the interior cavity of the housing;
a movable plug positioned within the interior cavity of the housing and configured to move into the one or more cartridges to eject the liquid suspension or aqueous solution of medication from the reservoir;
wherein the one or more cartridges each have an ejection nozzle and wherein the liquid suspension or aqueous solution of medication is ejected from the cartridge and the housing through the ejection nozzle, and
wherein the housing supports an alignment mechanism for automatically aligning a user's eye with the ejection nozzle.

2. The system of claim 1 and further comprising an actuator for initiating movement of the movable plug for ejecting the liquid suspension or aqueous solution of medication from the cartridge.

3. The system of claim 2 wherein the actuator is a linear actuator.

4. The system of claim 2 wherein the actuator is a piezoelectric actuator.

5. The system of claim 1 wherein the alignment mechanism comprises a pivotal arm coupled to the housing at a first end wherein a second end of the pivotal arm contacts a nose bridge of the user.

6. The system of claim 1 wherein the housing further supports a speaker thereon and electrical components for the speaker within the interior cavity and wherein the system is configured to provide an audible indicator or instruction for actuation of the system.

7. The system of claim 1 wherein the housing further supports a light source for providing a visual indicator for alignment or focus for actuation of the system.

8. The system of claim 1 wherein each of the one or more cartridges are disposable.

9. The system of claim 1 wherein each of the one or more cartridges comprise at least one, metered doses of medication for dosing each eye before replacement of the cartridge.

10. A cartridge-based device for providing a spritz of medication to a user's eyeball, the device supporting a sealed cartridge comprising at least two metered doses of medication wherein a moveable plug inside the device is actuated to force the medication through an ejection nozzle of the cartridge in the form of a spritz.

11. The device of claim 10 wherein the moveable plug provides the seal for the cartridge and wherein the movable plug facilitates the volumetric expansion and contraction of the medication allowing the cartridge to withstand various temperature and pressure changes as dictated by storage conditions, while maintaining the sterility of the medication therein.

12. The device of claim 10 and further comprising a pivotal alignment mechanism configured to contact a structure of the user's face, such as the nose bridge, brow, or eye socket, wherein the alignment mechanism spaces the ejection nozzle from the eye while maintaining alignment of the nozzle with the eye.

13. The device of claim 10 wherein the alignment mechanism comprises one or more sensors for detecting contact with the skin of the user for initiating actuation and movement of the movable plug.
